SEQUENCE LISTING (15 sequences)

SEQ ID NO: 1 - protein, 21 aa, Homo sapiens
MRAWIFFLLCLAGRALAAPLA

SEQ ID NO: 2 - protein, 8 aa, Artificial Sequence
  This is an artificial sequence used to aid in the purification process
DYKDDDDK

SEQ ID NO: 3 - protein, 92 aa, Homo sapiens
LAGSHSMRYFSAAVSRPGRGEPRFIAMGYVDDTQFVRFDSDSACPRMEPRAPWVEQEGPEYWEEETRNTKAHAQTDRMNLQTLRGYYNQSEA

SEQ ID NO: 4 - protein, 92 aa, Homo sapiens
SSHTLQWMIGCDLGSDGRLLRGYEQYAYDGKDYLALNEDLRSWTAADTAAQISKRKCEAANVAEQRRAYLEGTCVEWLHRYLENGKEMLQRA

SEQ ID NO: 5 - protein, 92 aa, Homo sapiens
DPPKTHVTHHPVFDYEATLRCWALGFYPAEIILTWQRDGEDQTQDVELVETRPAGDGTFQKWAAVVVPSGEEQRYTCHVQHEGLPEPLMLRW

SEQ ID NO: 6 - protein, 21 aa, Homo sapiens
SKEGDGGIMSVRESRSLSEDL

SEQ ID NO: 7 - protein, 326 aa, Homo sapiens
MRAWIFFLLCLAGRALAAPLADYKDDDDKLAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 8 - protein, 234 aa, Homo sapiens
MRAWIFFLLCLAGRALAAPLADYKDDDDKLAGSHSMRYFSAAVSRPGRGEPRFIAMGYVDDTQFVRFDSDSACPRMEPRAPWVEQEGPEYWEEETRNTKAHAQTDRMNLQTLRGYYNQSEANPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 9 - DNA, 20 bp, Artificial Sequence
  This is a forward primer that matches the coding region for the signal peptide
gccctgaccctgaccgagac

SEQ ID NO: 10 - DNA, 18 bp, Artificial Sequence
  This is a reverse primer that matches the 3′ untranslated regions of the transcripts
tgagacagacggagacat

SEQ ID NO: 11 - DNA, 24 bp, Artificial Sequence
  This is a 5′ phosphorylated cloning primer
ggctcccactccatgaggtatttc

SEQ ID NO: 12 - DNA, 24 bp, Artificial Sequence
  This is the cloning primer which includes the stop codon and part of the intron4
gggtctagattaaaggtcttcaga

SEQ ID NO: 13 - DNA, 26 bp, Artificial Sequence
  This is a primer sequence
ctagctagctagaggctcccactcca

SEQ ID NO: 14 - DNA, 30 bp, Artificial Sequence
  This is a primer sequence
ctagtctagactagattaaaggtcttcaga

SEQ ID NO: 15 - DNA, 22 bp, Artificial Sequence
  This is a primer sequence
gcatcatgtctgttagggaaag

(19) United States
(12) Patent Application Publication
(10) Pub. No.: US 2004/0044182 A1
(43) Pub. Date: Mar. 4, 2004

(54) Expression, preparation,uses, and sequence of recombinantly-derived soluble hla-g

(76) Inventors: Joan S Hunt, Shawnee Mission, KS (US); Pedro J. Morales, Kansas City, MO (US); Margaret G. Pertroff, Merriam, KS (US)

Correspondence Address: Hovey Williams, 2405 Grand Blvd, Suite400, Kansas City, MO, 64108, US

(21) Appl. No.: 10/380,880

(22) PCT Filed: Sep. 17, 2001
(86) PCT No.: PCT/US01/29228

(51) Int. Cl. C07K 14/74; C07H 21/04

(52) U.S. Cl. 530/350; 435/69.1; 435/320.1; 435/325; 536/23.5

(57) ABSTRACT

Methods of producing and using recombinant soluble HLA-G are provided. This recombinant soluble HLA-G alters immune responses to tissues, organs, fetuses, and embryos which are genetically distinct from the organism receiving or possessing such antigenic material. Preferable forms of this protein include sequences having at least 70% sequence homology with naturally occurring forms of HLA-G. Specifically, each recombinant form of HLA-G produced and used by the present methods must include a sequence having at least 70% sequence homology to introm 4 expressed by the HLA-G gene and at least one sequence having at least 70% sequence homology to one of the α domains expressed by the HLA-G gene. Preferable forms of the present invention include one isoform which includes the α1 domain, the α2 domain, the α3 domain, and intron 4, and a second isoform which includes the α1 domain, the α3 domain and intron 4. Still more preferably, the sequences include a purification-assisting peptide sequence and a signal peptide.

### SEQUENCE LISTING

A printed sequence listing accompanies this application, and has also been submitted with identical contents in the form of a computer-readable ASCII file on CDROM.

### BACKGROUND OF THE INVENTION

1. Field of the Invention

The present invention is concerned with mechanisms affecting immune tolerance of organs, tissues, and cells having a genetically different origin than their host. More particularly, the present invention is concerned with immune tolerance of genetically different fetuses during pregnancy, genetically different organs which have been transplanted, and genetically different tissues or cells which have been grafted into an organism. More particularly, the present invention is concerned with recombinant proteins and methods of generating these recombinant proteins wherein the recombinant proteins affect tolerance of these foreign tissues, organs and cells. Still more particularly, the present invention is concerned with the use and production of recombinant soluble HLA-G protein including HLA-G1 and HLA-G2. Finally, and most particularly, the present invention is concerned with producing large amounts of recombinant soluble HLA-G1 and recombinant soluble HLA-G2 protein and using this recombinant protein to alter immune-initiated responses to genetically different fetuses or genetically different organs, tissues, and cells which have been implanted or grafted into an individual.

2. Description of the Prior Art

It has been determined that a soluble substance, soluble HLA-G, is synthesized in the placenta and circulates in mothers' blood throughout pregnancy. HLA-G is present at high concentrations at the maternal-fetal interface.

HLA-G is an MHC class Ib protein that has been identified as a product of human trophoblast cells. Subpopulations of these cells, which comprise the fetal compartment of the maternal-fetal interface, are unique in their substitution of class Ib antigens, HLA-G and HLA-E, which have few alleles, for the highly polymorphic HLA-A and HLA-B antigens. This substitution is believed to have a major role in the tolerance and accommodation of the genetically different embryo/fetus. This is because expression of class I genes such as HLA-E and HLA-G are unlikely to elicit an immune response due to a high degree of sequence conservation between all people which usually will not permit the immune system to distinguish between self and non-self antigens. This is contrast to genes encoding HLA class Ia antigens such as HLA-A and HLA-B. These genes express proteins which are highly polymorphic and which thereby invoke an immune response.

The changes from the patterns expressed by cells derived from the inner cell mass, which exhibit HLA-A and HLA-B, are thought to account in large part for maternal tolerance and accommodation of a genetically different embryo/fetus. The trophoblasts also express HLA-C but the significance of this is as yet unknown.

The HLA-G message is differentially spliced to produce at least six different transcripts. Three of the messages, HLA-G1, HLA-G2 and HLA-G3, encode isoforms with transmembrane domains and are therefore likely to exist primarily as membrane-bound proteins. These membrane-bound isoforms of HLA-G are believed to protect fetal trophoblast cells from attack by maternal macrophages and/or natural killer (NK) cells, both of which are present in substantial numbers in the specialized tissue derived from the uterine endometrium termed the decidua.

Two of the transcripts encode soluble isoforms that structurally resemble membrane-bound HLA-G1 and HLA-G2 but lack transmembrane domains due to reading of the messages into intron 4, which contains a stop codon. Soluble HLA-G1 (sHLA-G1) is composed of sequences from the α1, α2, and α3 domains followed by 21 amino acids from intron 4. Similarly, sHLA-G2 is identical to sHLA-G1 except that it lacks the α2 domain.

HLA-G has been partially purified from placentas and trophoblast tumor cell lines. However, purification of the natural protein does not result in an adequate supply of sHLA-G. Accordingly, what is needed is a method of producing unlimited quantities of pure recombinant proteins. Preferably, these proteins would be produced in human cells in order to assure correct glycosylation. Still more preferably, the recombinant proteins will be secreted from the cells and into the surrounding culture medium. What is further needed is a method of isolating and purifying these recombinant proteins from cell culture medium so that the cells will remain to produce these unlimited quantities of the recombinant soluble protein. What is still further needed are methods of altering immune response in pregnant individuals as well as in individuals undergoing organ transplants, and tissue and/or cell grafts.

### SUMMARY OF THE INVENTION

The present invention solves the problems inherent in the prior art and provides a distinct advance in the state of the art. By using methods of the present invention, unlimited quantities of recombinant soluble HLA-G protein can be produced and used to modulate immune responses in individuals. These recombinant proteins are produced in human cells thereby assuring proper glycosylation. Furthermore, these proteins give every evidence of identity with natural sHLA-G1 and sHLA-G2.

The recombinant proteins provided by the present invention have differing effects on different target cells. For example, recombinant soluble HLA-G1 (rsHLA-G1) may effect leukocytes and decidual cells in opposite ways and recombinant soluble HLA-G2 (rsHLA-G2) may effect these same cells differently than rsHLA-G1. Furthermore, excess amounts of rsHLA-G may have adverse effects on certain individuals or under certain conditions. It has also been found that HLA-G is present in men as well as women. However, it is apparent that HLA-G modulates immune response and that the recombinant proteins of the present invention hold great promise in the treatment of immune disorders and in other areas where modulation of the immune response is important.

It is believed that the present invention describes the first successful expression of rsHLA-G1 and rsHLA-G2 by eukaryotic cells. Such production is difficult in that the recombinant isoforms are difficult to prepare because they are derived from specific messages generated by alternative splicing of a single mRNA. The splice variants must be isolated from one another, sequenced, spliced into appropriate plasmids and transfected into human cells for production. Such a sequence of events is schematically illustrated in FIGS. 7, 8, and 9. The first of these figures, FIG. 7, illustrates soluble HLA-G1 and soluble HLA-G2 cloning into the expression vector pcDNA3.1HisC and expression of sHLA-G1 and sHLA-G2 as N-tagged proteins to 6x-His and Xpress. FIG. 8 illustrates the sub-cloning of sHLA-G1 and sHLA-G2 into pRC/CMV vector containing BM40 signal peptide for the expression of soluble and secreted HLA-G1 and HLA-G2 N-tagged to FLAG peptide. Finally, FIG. 9 illustrates the stable transfection of a preferred cell line (HEK293 cells) with ssHLA-G constructs (secreted products) and protein purification using FLAG-M2 affinity gel. A photograph of a Western Blot showing the proteins expressed by these transfected cells is given in FIG. 10.

In this application, sequences have been assigned the following SEQ ID NOS: the BM40 signal peptide is SEQ ID NO. 1; the Flag peptide is SEQ ID NO. 2; the α1 domain is SEQ ID NO. 3; the α2 domain is SEQ ID NO. 4; the α3 domain is SEQ ID NO. 5; intron 4 is SEQ ID NO. 6; recombinant soluble HLA-G1 (rsHLA-G1) is SEQ ID NO. 7; and recombinant soluble HLA-G2 (rsHLA-G2) is SEQ ID NO. 8. Sequences including or having a sequence which has at least about 70% sequence identity or homology with any one of SEQ ID NOS. 1-8 and which exhibit similar desired properties are within the scope of the present invention. Preferably, such sequences will have at least about 79% sequence identity or homology with any one of SEQ ID NOS. 1-8 and still more preferably at least about 90% sequence identity or homology. Most preferably, such sequences will have at least about 95% sequence identity or sequence homology with any one of SEQ ID NOS 1-8. Additionally, sequences which differ from any one of SEQ ID Nos. 1-8 due to a mutation event or series of mutation events but which still exhibit similar properties are also embraced in the present invention. Such mutation events include but are not limited to point mutations, deletions, insertions and rearrangements.

As used in this application, the following definitions will apply: “Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. et al., eds., M. Stockton Press, New York (1991); and Carillo, H., et al. Applied Math., 48:1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410(1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 95% “sequence identity” to a reference nucleotide sequence, it is intended that the nucleotide sequence of the given polynucleotide is identical to the reference sequence except that the given polynucleotide sequence may include up to 5 point mutations per each 100 nucleotides of the reference nucleotide sequence. In other words, in a polynucleotide having a nucleotide sequence having at least 95% identity relative to the reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 95% sequence identity to a reference amino acid sequence, it is intended that the given amino acid sequence of the polypeptide is identical to the reference sequence except that the given polypeptide sequence may include up to 5 amino acid alterations per each 100 amino acids of the reference amino acid sequence. In other words, to obtain a given polypeptide sequence having at least 95% sequence identity with a reference amino acid sequence, up to 5% of the amino acid residues in the reference sequence may be deleted or substituted with another amino acid, or a number of amino acids up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

Similarly, “sequence homology”, as used herein, also refers to a method of determining the relatedness of two sequences. To determine sequence homology, two or more sequences are optimally aligned as described above, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 95% of the amino acid residues or nucleotides in the reference sequence must match or comprise a conservative substitution with another amino acid or nucleotide, or a number of amino acids or nucleotides up to 5% of the total amino acid residues or nucleotides, not including conservative substitutions, in the reference sequence may be inserted into the reference sequence.

A “conservative substitution” refers to the substitution of an amino acid residue or nucleotide with another amino acid residue or nucleotide having similar characteristics or properties including size, charge, hydrophobicity, etc., such that the overall functionality does not change significantly.

Isolated” means altered “by the hand of man” from its natural state., i.e., if it occurs in nature, it has been changed or removed from its original environment, or both. For example, a polynucleotide or polypeptide naturally present in a living organism is not “isolated,” but the same polynucleotide or polypeptide separated from the coexisting materials of its natural state is “isolated”, as the term is employed herein. Finally, all references and teachings cited herein which have not been expressly incorporated by reference are hereby incorporated by reference.

“Stable” in terms of stable transfectants refers to the ability of a cell to continually produce or express a particular protein without damaging the cell. Stable transfectants will continue to express the protein indefinitely as long as the cells themselves are properly maintained.

A “signal peptide” refers to a peptide located at the N-terminal end of a protein which assist the protein in becoming incorporated into or transported and secreted through the plasma membrane of a cell. Post translational cleavage usually removes these signal sequences from the respective precursor protein.

Additionally, this application claims the benefit of a prior provisional application (Application S/No. 60/232,761), the content and teachings of which are hereby incorporated by reference herein.

The teachings and content of the following references are also incorporated by reference herein.:

U.S. Pat. No. 5,856,442, Inventor: Carosella, Edgardo et al., Transcripts of the MHC Class I HLA-G Gene and Their Applications; Issued Jan. 5, 1999

Chu, et al. (1998) Soluble HLA-G in human placentas: synthesis in trophoblasts and interferon-y-activated macrophages but not placental fibroblasts. Human Immunol., 59:435-442

Ellis, et al. (1990). Human trophoblast and the choriocarcinoma cell line BeWo express a truncated HLA class I molecule. J. Immunol., 144:731

Fujii, et al. A soluble form of the HLA-G antigen is encoded by a messenger ribonucleic acid containing intron 4. J. Immunol., 153:5516 (1994)

Hunt, et al. Expression of class I HLA genes by trophoblast cells: analysis by an in situ hybridization. J. Imunol., 140:1293-1299 (1988)

Hunt, Joan, Major histocompatibility antigens in reproduction. 392-402

Hunt, J., et al., HLA-G in Reproduction: Studies on the Maternal-Fetal Interface. Hum. Immun. 61, 113-117 (2000)

Hunt, J., et al., Soluble HLA-G circulates in maternal blood during pregnancy. Am. J. Obstet. Gynecol, Volume 183, No. 3, 682-688 (2000)

Ishitani et al. (1992). Alternative splicing of HLA-G transcripts yields proteins with primary structures resembling both class I and class II antigens. Proc. Natl. Acad. Sci. USA, 89:3947

Lila, M., et al., Implication of HLA-G molecule in heart-graft acceptance. The Lancet, Vol. 355 (2000)

Rouas-Freiss, N., et al., The α₁ domain of HLA-G1 and HLA-G2 inhibits cytotoxicity induced by natural killers cells: Is HLA-G the public ligand for natural killer cell inhibitory receptors? Proc. Natl. Acad. Sci. USA, Vol. 94, 5249-5254, (1997)

Rouas-Freiss, N., et al. Direct evidence to support the role of HLA-G in protecting the fetus from maternal uterine natural killer cytolysis. Proc. Natl. Acad. Sci. USA Vol. 94, 11520-11525, (1997).

Rouas-Freiss, N., et al. The immunotolerance role of HLA-G. Cancer Biology, Vol. 9, 3-12 (1999).

In one aspect of the present invention, a method of producing recombinant soluble HLA-G is provided. The method includes the steps of transfecting a cell with a recombinant expression vector wherein the recombinant expression vector includes a sequence encoding for intron 4 of HLA-G. Additionally, the recombinant expression vector includes at least one sequence which encodes for a domain of HLA-G. These HLA-G domains include the α1 domain, the α2 domain, the α3 domain and combinations of these domains. When more than one of these domains is included, the sequences encoding for the domains preferably appear in numerical order. That is to say that the α1 domain will preferably precede the α2 domain which will preferably precede the α3 domain. The sequence of the recombinant expression vector is then expressed in the cell. When the recombinant expression vector includes a sequence encoding for a signal peptide, the recombinant protein can be secreted from the cell, into the culture medium, whereupon it may be isolated and purified. The expressed recombinant protein preferably has a sequence having at least 70% sequence homology with a sequence selected from the group consisting of SEQ ID NOS. 7 and 8. Still more preferably, the recombinant protein has at least 79% sequence homology and still more preferably at least 90% sequence homology with a sequence selected from the group consisting of SEQ ID NOS. 7 and 8. Most preferably, the expressed recombinant protein has at least 95% sequence homology with either SEQ ID NO. 7 or SEQ ID NO. 8.

In another aspect of the present invention a method for obtaining recombinant soluble HLA-G protein is provided. This method includes the steps of transfecting a first cell containing the gene encoding HLA-G protein;isolating RNA from said transfected cell;preparing cDNA from said isolated RNA; amplifying said cDNA by performing PCR on said cDNA; selecting for amplified cDNA containing selected portions of the cDNA encoding for HLA-G; ligating said amplified cDNA containing said selected portions into a vector to produce a recombinant expression vector; transfecting a second cell with said recombinant expression vector; and expressing said selected portions using said transfected second cell. In preferred forms, the first cell comprises a mouse fibroblast cell line and a particularly preferred mouse fibroblast cell line is denominated S14/8. In order to prepare cDNA from the isolated RNA, it is preferred to use reverse transcriptase with a particularly preferred reverse transcriptase being Moloney Murine Leukemia Virus Reverse Transcriptase. When amplifying the cDNA using PCR, it is preferred to use a forward primer comprising a sequence having at least about 70% sequence homology with SEQ ID NO. 9. A preferred reverse primer comprises a sequence having at least about 70% sequence homology with SEQ ID NO. 10. The selected portions of amplified cDNA preferably include sequences encoding for sequences having at least 70% sequence homology with SEQ ID NO. 6 coupled with at least one sequence having at least 70% sequence homology with a sequence selected from the group consisting of SEQ ID NOS. 3-5. More preferably, the sequences have at least 79% sequence homology and still more preferably at least 90% sequence homology with any of SEQ ID NOS. 3-6. The ligating step preferably utilizes T4 DNA ligase and the vector is preferably linearized. A particularly preferred expression vector is denominated pcDNA3.1HisC. For this method, the second cell is preferably a eukaryotic cell and still more preferably a mammalian cell. One preferred group of mammalian cells includes HEK293 cells, human trophoblasts tumor cells, and Chinese hamster ovary cells. Particularly preferred among this group are the HEK293 cells. The expressed selected portions preferably comprise recombinant soluble HLA-G protein. This recombinant soluble protein is preferably selected from the group consisting of HLA-G1 and HLA-G2, and still more preferably has at least 70% sequence homology with either SEQ ID NO. 7 or SEQ ID NO. 8.

In another aspect of the present invention, a method of promoting the acceptance of a fetus by the immune system of an individual is provided. This method comprises the steps of administering a recombinant protein to the individual and altering cytokine production in the individual by contacting a target cell within the individual with the recombinant protein. Preferably, the recombinant protein has at least 70% sequence homology with an amino acid sequence wherein the amino acid sequence comprises SEQ ID NO. 6 and at least one sequence selected from the group consisting of SEQ ID NOS. 3-5. The recombinant protein may also include a sequence selected from the group consisting of SEQ ID NO. 1, SEQ ID NO. 2, and combinations thereof. Alternatively, the recombinant protein may comprise an amino acid sequence having at least 70% sequence homology with a sequence selected from the group consisting of SEQ ID NOS. 7 and 8. It is also preferably for the recombinant protein to be soluble in aqueous solutions. Such solutions should comprise at least 50% water and may include at least one member selected from the group consisting of salts, detergents, and buffers. Altering cytokine production can be accomplished in various ways including the alteration of signal transduction in the target cell. Preferably, the target cell is capable of binding soluble HLA-G and the target cell may be selected from the group consisting of leukocytes and decidual cells.

In another method of the present invention an individual's immune tolerance to foreign tissue is modulatable. The tissue may be organs, cells, or a tissue graft. The method generally comprises the steps of administering a recombinant protein to the individual wherein the recombinant protein binds with a target cell whereupon the target cell's gene expression is modulated. This modulation may result in an increase or a decrease in the gene expression. In terms of the present invention, it is preferable that the target cell be capable of binding with soluble HLA-G. Target cells include many cells including leukocytes and decidual cells. Similar to the above-described method for promoting acceptance of a fetus, it is preferred that the recombinant protein have at least 70% sequence homology with an amino acid sequence which comprises SEQ ID NO. 6 and at least one of SEQ ID NOS. 3-5. Such a sequence may further include SEQ ID NO. 1, SEQ ID NO. 2, or combinations thereof. Alternatively, the recombinant protein may comprise an amino acid sequence having at least 70% sequence homology with either SEQ ID NO. 7 or SEQ ID NO. 8. Preferably, the recombinant protein is soluble in aqueous solutions. Such aqueous solutions include solutions comprising at least 50% water and may include at least one member selected from the group consisting of salts, detergents, and buffers. One of the preferred ways to modulate the gene expression of the target cell includes the step of altering signal transduction in the target cell.

The recombinant protein of the present invention may comprise a first amino acid sequence having at least 70% sequence homology with SEQ ID NO. 3, a second amino acid sequence linked with the first amino acid sequence and having at least 70% sequence homology with SEQ ID NO. 5, and a third amino acid sequence linked with the second amino acid sequence and having at least 70% sequence homology with SEQ ID NO. 6. Preferred forms of this recombinant protein further include a signal peptide. One preferred signal peptide has at least 70% sequence homology with SEQ ID NO. 1. Another preferred signal peptide is the BM40 signal peptide. It is also preferred to have the signal peptide linked with the first amino acid sequence. A fourth amino acid sequence may also be linked with the first amino acid sequence and it is preferred that this fourth amino acid sequence have at least 70% sequence homology with SEQ ID NO. 1. In yet another alternative but preferred form, the protein includes a fifth amino acid sequence which preferably has at least 70% sequence homology with SEQ ID NO. 2. Another alternative form attaches a sixth amino acid sequence to the first amino acid sequence wherein this sixth amino acid sequence preferably has at least 70% sequence homology with SEQ ID NO. 4. In all of the sequences listed in this paragraph, it is preferred for the sequences to have at least 79% sequence homology with its corresponding SEQ ID NO. Additionally, the recombinant protein should be soluble in aqueous solutions, as described above. Effective recombinant proteins are also capable of altering activity in a cell and such cells include peripheral blood mononuclear cells and decidual cells.

In another aspect of the present invention, a recombinant protein encoded by a gene transcript of a cell is provided. The transcript comprises, in succession from the 5′ end to the 3′ end, a first sequence encoding the α1 domain of the protein, a second sequence encoding the α3 domain of the protein and a third sequence encoding intron 4 of the protein. Preferably, the recombinant protein has at least 70% sequence homology with HLA-G. In preferred forms, the transcript further comprises a fourth sequence encoding the α2 domain of the protein. In other preferred forms the transcript further comprises a fifth sequence encoding a signal peptide. Preferably, this signal peptide is cleaved from the recombinant protein in the cell and permits the recombinant protein to be secreted into the culture media. In yet another preferred form, the transcript further comprises a sixth sequence which encodes a purification-assisting peptide. Preferably, this purification-assisting peptide is cleaved from the recombinant protein in the cell. Sequences having at least 70% sequence homology with SEQ ID NO. 2 are embraced by the present invention. An advantage of the present invention is that the recombinant protein maybe expressed in a eukaryotic cell. This eukaryotic cell is preferably a transfected mammalian cell and particularly preferred transfected mammalian cells are transfected with an expression vector for soluble HLA-G1 (sHLA-G1) and soluble HLA-G2 (sHLA-G2). Alternatively, the expression vector preferably includes a sequence encoding a protein having at least 70% sequence homology with either SEQ ID NO. 7 or SEQ ID NO. 8. The transfected cell may be from a variety of sources, however, particularly preferred cells include HEK293 cells, human trophoblast tumor cells, and Chinese hamster ovary cells. Of these, HEK293 cells are generally preferred due to their lack of a tendency to develop tumors. The recombinant protein should also be soluble in aqueous solutions, such as those described above.

In yet another aspect of the present invention, a recombinant protein is provided. This recombinant protein has at least 70% sequence homology with an amino acid sequence wherein the amino acid sequence comprises SEQ ID NO. 6 and at least one sequence selected from the group consisting of SEQ II) NOS. 3-5. This coincides with HLA-G recombinant proteins which are encoded by a transcript which includes intron 4 and at least one α domain. Other preferred recombinant proteins include an amino acid sequence selected from the group consisting of SEQ ID NO. 1, SEQ ID NO. 2, and combinations thereof. These two sequences provide the signal peptide (SEQ ID NO. 1) and the purification-assisting peptide (SEQ ID NO. 2). Of course, other signal peptides would be useful for purposes of the present invention as would other purification-assisting peptides. Preferred forms of the recombinant protein have at least 70% sequence homology with a first amino acid sequence which comprises, in succession, SEQ ID NO. 3, SEQ ID NO. 5, and SEQ ID NO. 6. Other forms of this preferred recombinant protein may include a second amino acid sequence which is linked to the first amino acid sequence and has at least 70% sequence homology with SEQ ID NO. 1. Yet another preferred recombinant protein comprises, in succession, amino acid sequences having at least 70% sequence homology with SEQ ID NO. 1, SEQ ID NO. 2, SEQ ID NO. 3, SEQ ID NO. 5, and SEQ ID NO. 6. Such a sequence corresponds to HLA-G2 and includes the signal peptide, the Flag peptide, the α1 doamin, the α3 domain and intron 4. Still another preferred recombinant protein comprises, in succession, amino acid sequences having at least 70% sequence homology with SEQ ID NO. 1, SEQ ID NO. 2, SEQ ID NO. 3, SEQ ID NO. 4, SEQ ID NO. 5, and SEQ ID NO. 6. Such a sequence corresponds to HLA-G1 and includes all of the portions of HLA-G2 and the α2 domain. Preferably, the protein is expressed by transfected eukaryotic cells. These transfected eukaryotic cells are preferably mammalian cells including cells selected from the group consisting of HEK293 cells, human trophoblast tumor cells, and Chinese hamster ovary cells. Of these cells, it is preferred to use HEK293 cells transfected with an expression vector which includes a gene encoding HLA-G. More preferably, the expression vector includes sequences encoding for sHLA-G1 or sHLA-G2. Other preferred recombinant proteins include amino acid sequences having at least 70% sequence homology with either SEQ ID NO. 7 or SEQ ID NO 8. Again, these recombinant proteins are preferably soluble in aqueous solutions with preferable aqueous solutions being described above.

The present invention also provides a recombinant expression vector wherein the vector expresses a recombinant protein having at least 70% sequence homology with an amino acid sequence which includes SEQ ID NO. 6 and at least one sequence selected from the group consisting of SEQ ID NOS. 3-5. Preferred forms of this recombinant expression vector express recombinant proteins which include amino acid sequences having at least 70% sequence homology with SEQ ID NO. 1, SEQ ID NO. 2, or combinations thereof. One particularly preferred recombinant protein expressed by the recombinant expression vector of the present invention has at least 70% sequence homology with a first amino acid sequence which comprises, in succession, SEQ ID NO. 3, SEQ ID NO. 5, and SEQ ID NO. 6. When coupled with SEQ ID NOS. 1 and 2, such a recombinant protein has at least 70% sequence homology with HLA-G2. Another particularly preferred recombinant protein expressed by the recombinant expression vector of the present invention has at least 70% sequence homology with a first amino acid sequence which comprises, in succession, SEQ ID NO. 3, SEQ ID NO. 4, SEQ ID NO. 5, and SEQ ID NO. 6. Such a recombinant protein has at least 70% sequence homology with HLA-G 1. It is preferred for the recombinant expression vector to be transfected into eukaryotic cells and that the resultant transfected eukaryotic cells be stable. Preferred eukaryotic cells include mammalian cells and preferred mammalian cells include HEK293 cells, human trophoblast tumor cells, and Chinese hamster ovary cells. Of course, it is preferred that the amino acid sequences making up the recombinant protein are expressed in the transfected cells and still more preferable for the recombinant protein to be secreted from the transfected cells. By secreting the recombinant protein, the cells may remain intact and continue producing additional recombinant protein. The presence of SEQ ID NO. 6 renders the recombinant protein soluble in aqueous solutions.

### BRIEF DESCRIPTION OF THE DRAWINGS

FIG. 1 is a schematic representation of a portion of the HLA-G gene (HLA 6.0) and the two alternatively spliced transcripts sHLA-G1 and sHLA-G2;

FIG. 2 is a schematic representation of the expected recombinant products and expected molecular weights;

FIG. 3 is schematic representation of ELISA results confirming that recombinant sHLA-G1 and recombinant sHLA-G2 were produced and secreted by the HEK293 clones;

FIG. 4 is a photograph of an immunoblot detecting the recombinant proteins sHLA-G1 and sHLA-G2 using two different antibodies;

FIG. 5 is a photograph of an immunoblot and a schematic representation of the immunoblot results comparing enzymatic deglycosylation of the purified recombinant proteins under native and denaturing conditions;

FIG. 6 is a photograph of an immunoblot testing the recombinant proteins for binding to β2 m;

FIG. 7 is a schematic representation of the cloning into the expression vector and expression of the soluble proteins;

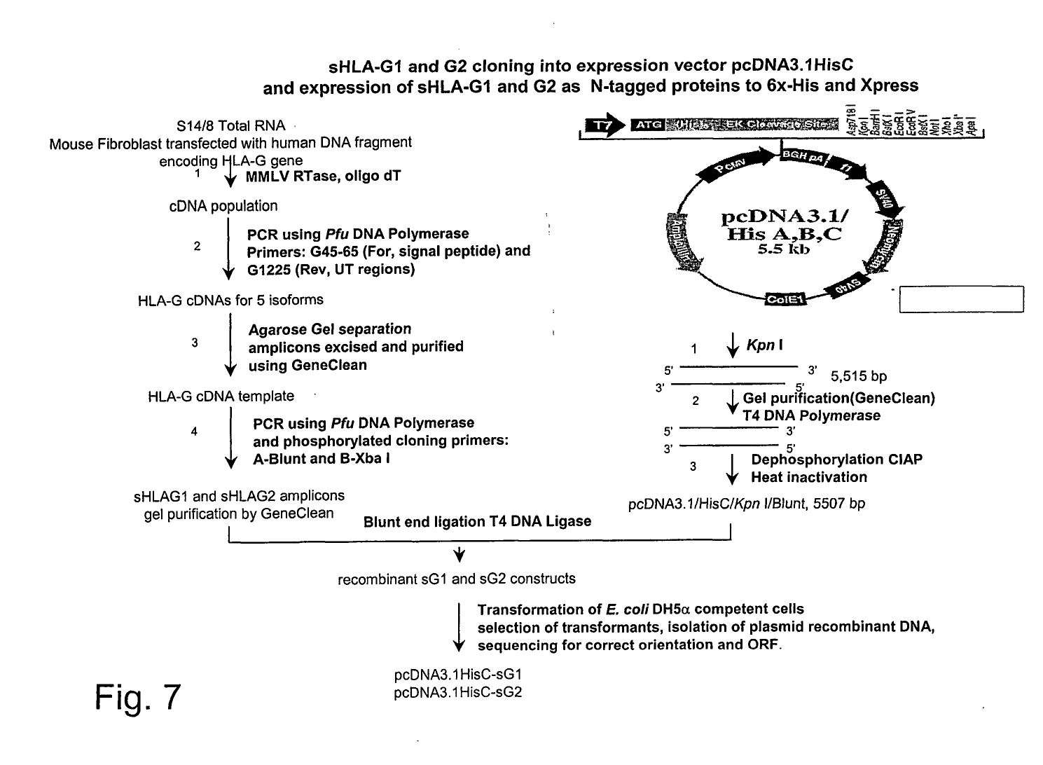

FIG. 8 is a schematic representation of the sub-cloning of the soluble proteins into the vector for the expression of the soluble and secreted proteins;

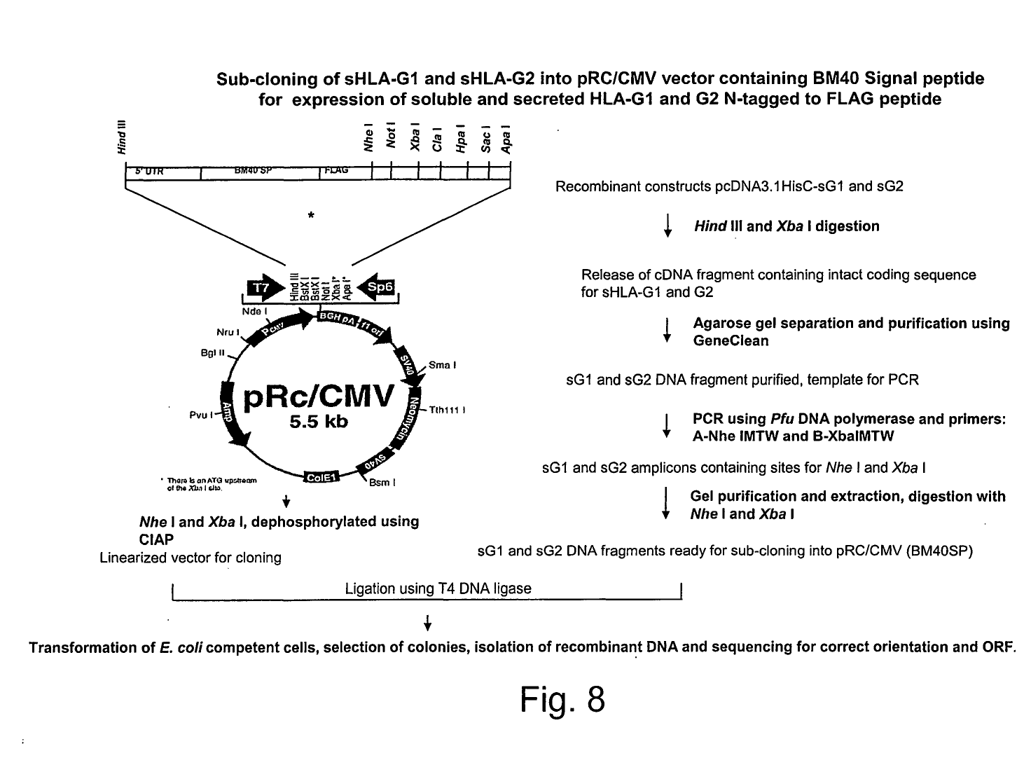

FIG. 9 is a schematic representation of the stable transfection of HEK293 cells with the secreted products and the protein purification used in the present invention;

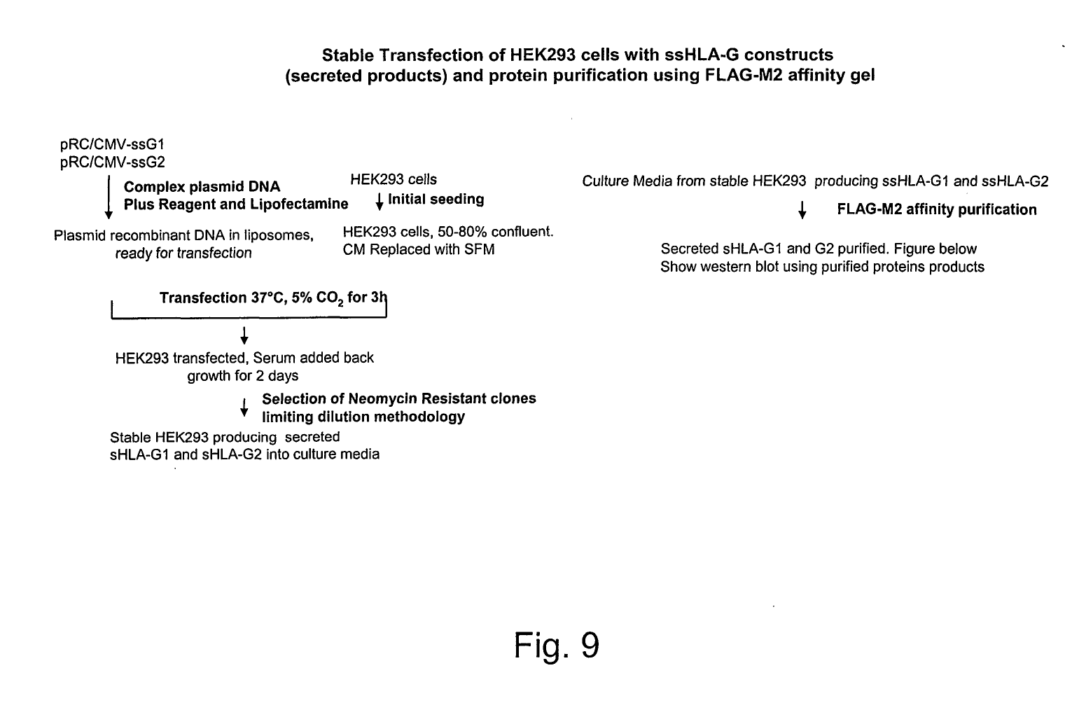

FIG. 10 is a Western Blot showing the purified proteins expressed by the transfected cells; and

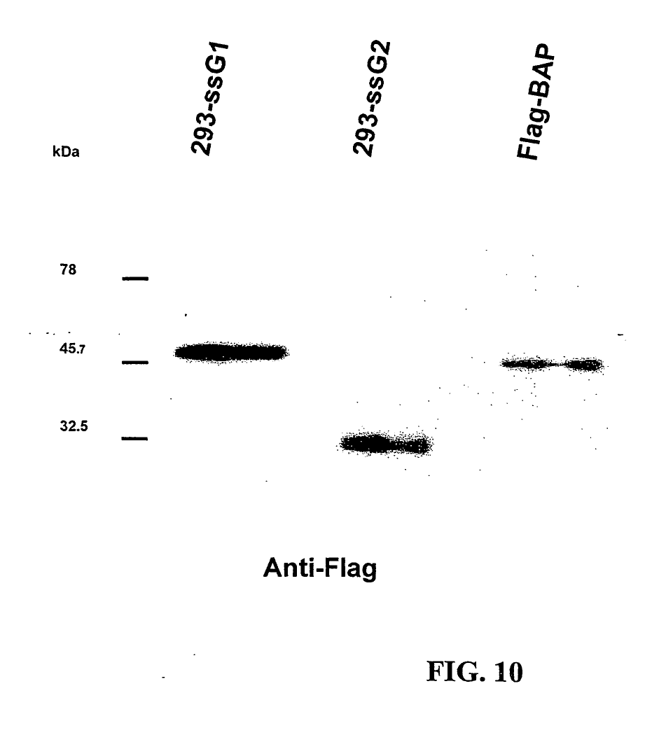

FIG. 11 is a graph showing IL-10 production in decidual macrophages treated with rsHLA-G.

### DETAILED DESCRIPTION OF THE PREFERRED EMBODIMENTS

The following examples set forth preferred methods in accordance with the invention. It is to be understood, however, that these examples are provided by way of illustration and nothing therein should be taken as a limitation upon the overall scope of the invention.

### EXAMPLE 1

This example maintained the cell lines used to generate clones and also generated the stably transfected clones.

Materials and Methods

Cells from the human embryonic kidney (HEK293) cells (ATCC No. CRL-1573) were selected for transfection with the construct resulting from the transfection of the S14/8.3 mouse fibrolblast cell line with the full length HLA 6.0 gene. The HEK293 and S14/8.3 cells were maintained at 37° C./5% CO₂ in Dulbecco's Modified Eagle Medium (D-MEM) supplemented with 10% fetal bovine serum (FBS) and penicillin (100U/ml)/streptomycin (0.1 mg/ml). Transfections were performed using LipofectAE Plus (Life Technologies, Grand Island, N.Y.) according to the manufacturer's specifications. Stable transfectants were selected using G418 (Life Technologies) and cloned by limiting dilution. Selection of positive sHLA-G producing clones was achieved by using an ELISA to test for the presence of the secreted protein in the culture media supematants.

### EXAMPLE 2

This example confirmed that the selected clones from Example 1 expressed the desired proteins.

Materials and Methods

Culture supernatants from the HEK293-sHLA-G1 and sHLA-G2 expressing clones and the negative control HEK293 stably transfected with the empty vector were collected after clones were grown for 3 to 9 days at an initial cell density of 2×10 6 cells per T75 flask. Protein production was monitored by an ELISA assay using a mouse monoclonal antibody specific for sHLA-G, 16G1 which is an antibody against intron 4 of HLA-G. Briefly, 50 μl of the HEK293 culture media supernatant in two duplicate wells for each primary antibody was added per well in a 96-well polystyrene plate (Corning Inc., Coming, N.Y.) and incubated overnight at 4C. to allow binding of the antigen. The next day, each well was washed twice with 150 μl of PBS per well and unoccupied sites of the wells were blocked with 200 μl per well of 5% non-fat dry milk (Bio-Rad, Hercules, Calif.) in PBS at room temperature for 30 minutes. After a single wash using 200 μl per well of wash buffer (0.05M Tris buffer pH 8.0, containing 0.15M NaCl and 0.05% Tween 20) each well was incubated with 50 μl of either 0.2 μl of 16G1 or control mouse IgG (Vector Laboratories, Burlingame, Calif.) in blocking solution at 37C. for 30 minutes. Subsequently, each well was washed four times with 200 μl of wash buffer, 50 μl/per well of 2 μg/ml of the secondary antibody, peroxidase labeled horse anti-mouse IgG (Vector Laboratories), was added and incubated at 37C. for 30 minutes. Unbound secondary antibody was released by washing four times with washbuffer and the substrate forperoxidase, TMB (Kirkegaard & Perry, Gaithersburg, Md.) was added. The plate was incubated at room temperature for color development for 5 to 15 minutes, at which time the reaction was stopped by adding 50 μl/per well of 1M H3Po4. The color reactions were read at 450 nm with an E₁ₓ 808 microplate reader (Bio-Tek Instruments, Inc, Winnoski, Vt).

### EXAMPLE 3

This example purified the isolated RNA and performed RT-PCR in order to obtain sHLA-G cDNAs.

Materials and Methods

Total RNA was isolated from S14/8.3 cells using TRIzol Reagent (Life Technologies, Gaithersburg, Md.) in accordance with the manufacturer's instructions. One microgram of heat denatured RNA (55 to 60C. for 10 min) from S14/8.3 cells was used to prepare cDNA using oligo dT₁₂₋₁₈ primer and Moloney Murine Leukemia Virus (MMLV) reverse transcriptase (Life Technologies) according to the manufacturer's procedure. Four microliters of cDNA were subjected to PCR in 50 μl reaction to obtain all of the alternatively spliced transcripts for HLA-G using a forward primer that matches the coding region for the signal peptide, G45-65: 5′-GCC CTG ACC CTG ACC GAG AC-3′ (SEQ ID NO. 9), and a reverse primer that matches the 3′untranslated regions of the transcripts, G1225: 5′-TGA GAC AGA CGG AGA CAT-3′ (SEQ ID NO. 10). To ensure high fidelity of the sequence for each amplicon, pfu DNA polymerase (Stratagene, La Jolla, Calif.) was used for PCR. Amplicons were separated by agarose gel electrophoresis, eluted from the gel and purified using Geneclean (Bio 101, Vista, Calif.). The presence of intron 4 in each amplicon was verified by PCR using previously described primers Gsi4 (SEQ ID NO. 15) and G1225 (SEQ ID NO. 10). Purified amplicons containing intron 4 were used as a templates in nested PCR using the 5′-phosphorylated cloning primers A-blunt: 5′-GGCTCC CAC TCC ATG AGG TAT TTC-3′ (SEQ ID NO. 11), which includes the beginning of the α1 domain coding region, and B-Xba: I5′-GGG TCT AGA TTA AAG GTC TTC AGA-3′ (SEQ ID No. 12), which includes the stop codon and part of intron 4. Two amplicons of approximately 900 and 600 bp were obtained, which were then separated by agarose gel electrophoresis and purified by Geneclean.

### EXAMPLE 4

This example constructed the plasmid containing the expression vector.

Materials and Methods

The mammalian expression vector pcDNA3.1HisC (Invitrogen, Carlsbad, Calif.) was linearized using the restriction enzyme KpnI and gel purified as described previously. To generate blunt ends the resulting 3′ protuberant ends were digested using the 3′→5′ exonuclease activity of T4 DNA polymerase (Life Technologies) and subsequently the vector was dephosphorylated using calf alkaline phosphatase (Life Technologies) to reduce self re-ligation. Using T4 DNA ligase (Life Technologies), purified cDNA for sHLA-G1 and sHLA-G2 were ligated into pcDNA3.1HisC modified as described previously. The constructs, named pcDNA3.1HisC-sG1and -sG2 generated are N-tagged to the Xpress™ epitope and 6xHis tag and their products are not secreted. For secreted products, the fragment HindIII/XbaI was released from the constructs containing the intact cDNA for sHLA-G1 and sHLA-G2. PCR was performed using these purified fragments as a template with the modified primers A-NheIMTW 5′-CTA GCT AGC TAG AGG CTC CCA CTC CA-3′ (SEQ ID NO. 13) and B-XbaIMTW 5′-CTA GTC TAG ACT AGA TTA AAG GTC TTC AGA-3′ (SEQ ID NO. 14). Each generated amplicon containing NheI and XbaI sites was gel purified by Geneclean and digested with the restriction enzymes then cloned into NheI and XbaI sites of the modified vector pRc/CMV containing the BM40 signal peptide. This expression vector, derived from pRc/CMV (Invitrogen), contains a HindIII-ApaI DNA fragment encoding the signal peptide of the basal membrane protein (BM40) along with the Flag peptide and a site for enterokinase cleavage within the poly-linker site of the vector. The sequences of the expression constructs were determined by the flourescent dideoxy-terminator chemistry method, using an ABI Prism™ DNA sequencing kit (PE Applied Biosystems, Foster City, Calif.) following the manufacturer's protocol. The sequences were analyzed using an ABI automated sequencer (ABI377 Prism™, PE Applied Biosystems). Sequence alignments with the HLA-G 6.0 gene (GeneBank Acc. No. J03027) were performed with MegAlign Lasergene software (DNASTAR Inc., Madison, Wis.).

### EXAMPLE 5

This example used immuno-affinity purification to select for the Flag-tagged recombinant sHLA-G1 and sHLA-G2 using Flag M2 affinity gel chromatography.

Materials and Methods

Culture media supernatants from HEK293-sHLA-G1 and -sHLA-G2 clones were collected after 3 to 9 days in culture, centrifuged to pellet any cell debris and then filter-sterilized using a 0.22 micron polyethersulphone filter (Corning, New York, N.Y.). The filter-sterilized culture media supernatant was loaded into a 2 ml packed equilibrated Flag-M2 agarose bead affinity chromatography column (Sigma-Aldrich, St. Louis, Mo.). All devices and instrumentation were handled aseptically to avoid contamination, and endotoxin-free water (Baxter Healthcare Corporation, Deerfield Ill.) was used for all solutions. The resin bed was washed with 3 bed volumes of TBS (50 mM Tris-HCl, 150 mM NaCl, pH 7.4) after the culture media supernatant entered the resin bed. The recombinant proteins were subsequently eluted under native conditions by competition with the Flag peptide (SEQ ID NO. 2) (Sigma-Aldrich) in TBS at 100 μg/ml. Aliquots of 0.5 ml were collected and analyzed by SDS-PAGE. The aliquots containing the protein were pooled and subjected to ultra-filtration using Ultrafree®-4 (Millipore, Bedford, Mass.) to concentrate, exchange buffer for PBS and separate the purified protein from the Flag peptide. Subsequently, the recombinant proteins were filter sterilized by using 0.22 μm low protein binding filter devices (Millex-GV4, Millipore) and subjected to endotoxin detection by Pyrotell® gel-clot formulation Limulus amebocyte lysate assay, detection limit 0.03 EU/ml (Associates of Cape Cod Inc., Falmouth, Mass.) to verify the absence of bacterial contamination during the purification steps. The purity was determined to be greater than 90% by Coomasie blue staining and densitometric analysis using a Gel-Pro Analyzer (version 3.0, Media Cybernetics).

### EXAMPLE 6

This example detected the soluble forms of HLA-G and β2 m using immunoblotting.

Materials and Methods

The purified recombinant proteins were resuspended in Laemmli buffer under reducing conditions and separated by electrophoresis in acrylamide gels (10% or 15% SDS- PAGE). The proteins were then electro-transferred onto nitrocellulose membranes (Schleider & Schuell, Keene, N.H.) and sHLA-G was detected using the monoclonal mouse antibody, 16G1 or anti-Flag M1 antibody (Sigma-Aldrich, St. Louis Mo.). The membrane subsequently was incubated with a peroxidase conjugated anti-mouse IgG antibody (Jackson inmuno Research Inc., West Grove, Pa.). The signal was developed using the chemiluminescent substrate SuperSignal West Pico (Pierce, Rockford, Ill.) and detected by exposing to Hyperfilm ECL™ (Amersham Pharmacia Biotech, Piscataway, N.J.). To determine whether the recombinant proteins bind β2 m, increasing amounts of the purified protein were loaded under reducing and denaturing conditions on a 15% SDS-PAGE gel, electro-transferred to nitrocellulose membranes and incubated with a mouse antibody specific for human β2 m (Amac Inc., Westbrook, Me.) at 1 μg/ml, and detected as described above.

### EXAMPLE 7

This example determined the glycosylation patterns of the purified recombinant sHLA-G1 and sHLA-G2.

Materials and Methods

To determine glycosylation patterns, purified recombinant sHLA-G1 and sHLA-G2 were digested under native and denaturing conditions using an enzymatic deglycosylation kit that cleaves N-linked and sialic acid-substituted Gal β1-3 GalNAc α1 O-linked oligosaccharides from glycoproteins (Bio-Rad, Hercules, Calif.). After enzymatic digestion, the samples were denatured in reducing Laemmli buffer, separated on SDS-PAGE acrylamide gels, transferred onto nitrocellulose membrane and detected with 16G1 antibody as described above.

### EXAMPLE 8

This example isolated peripheral blood mononuclear cells (PBMCs) which were later used to establish the biological activity of the recombinant HLA-G proteins.

Materials and Methods

Forty ml of blood was collected from a healthy male volunteer in accordance with approved protocols. The blood was diluted two-fold with RPMI medium (Mediatech, Herndon, Va.), layered over Histopaque-1077 (Sigma-Aldrich), and centrifuged for 30 minutes at 400×g. The enriched PBMC were washed, counted, and diluted to 1×10⁶ cells/ml in culture medium supplemented with 10% FBS (Atlanta Biologicals, Norcross, Ga.), 2 mM L-glutamine, 100 U/ml penicillin, and 100 mg/ml streptomycin (Sigma). To establish biological activity of the rsHLA-G1 and rsHLA-G2,PBMC were cultured in the absence or presence of recombinant human interferon IFN-γ (100 U/ml; Genzyme Diagnostics, Cambridge Mass.) and treated with PBS, rsHLA-G1 (1 μg/ml), or rsHLA-G2 (1 μg/ml) for six hours at 37C. in a 5% CO₂ incubator. The cells were then lysed in Trizol reagent for RNA extraction.

### EXAMPLE 9

This example extracted RNA from the cultured PBMC and prepared cDNA probes.

Materials and Methods

Total cellular RNA was extracted from the cultured PBMC using TRIzol Reagent (GibcoBRL, Grand Island, N.Y.) according to the manufacturer's instructions. The RNA was quantified by spectrophotometry, and 10 μg were treated with 4 units of DNase I (Sigma) to remove contaminating genomic DNA. For preparation of a cDNA probe, human cytokine cDNA labeling primers (Sigma-Genosys, Inc., The Woodlands, Tex.) were annealed to 1 μg of total RNA at 90C. for 2 minutes, followed by cooling to 42C. over a period of 20 minutes. Complementary DNA was then synthesized using 200U/ml of MMLV reverse transcriptase from the Strip-EZ RT StripAble cDNA Probe Synthesis and Removal Kit (Ambion, Austin, Tex.) in the presence of 20 mCi [α-³³P]-dATP (Perkin-Elmer Life Sciences, Boston, Mass.) according to the manufacture's instructions. Unincorporated nucleotides were removed from labeled cDNA using Bio-Spin 30 chromatography columns (Bio-Rad). A hand-held Geiger-Mueller counter was used to determine the percentage incorporation of the radioactive nucleotides into the cDNA. Incorporation of the radiolabeled nucleotide into the cDNA probes for the rsHLAG1 array, were control PBS, 40%, rsHLA-G1 treatment, 46%. For the rsHLA-G2 array incorporations were, 47% and 32% for the control PBS probe and rsHLAG2 probe, respectively. The cDNA probes had 75% (IFN-γ) and 45% incorporation (IFN-γ+rsHLA-G1) and 55% (IFN-γ) and 26% incorporation (IFN-γ+rsHLA-G2) for experiments testing the effect of rsHLA-G1 and rsHLA-G2 respectively on IFN-γ-activated PBMC.

### EXAMPLE 10

This example determined gene expression using a cDNA array analysis performed on PBMCs after incubation with recombinant soluble HLA-G proteins.

Materials and Methods

Panorama™ Human Cytokine Arrays (Sigma-Genosys) enable the analysis of 375 differentially expressed genes including cytokines and related factors, their receptors and housekeeping genes. Two Panorama Cytokine Array membranes (Sigma-Genosys) were pre-hybridized for at least 1 hour at 65C. in Panorama hybridization solution (Sigma-Genosys) containing 0.1 mg/ml salmon testes DNA (Sigma). The labeled cDNA synthesized from 1 μg of RNA from control, rsHLA-G1, or rsHLA-G2-treated PBMC was added to hybridization solution containing 0.1 mg/ml salmon testes DNA (Sigma) and denatured at 90C. to 95C. for 10 minutes. The arrays were hybridized overnight at 65C. and subsequently washed three times at room temperature in 0.5% SSPE, 1% SDS, followed by two washes at 65C. in 0.1% SSPE, 1% SDS. The arrays were exposed to Cyclone Storage phosphor screens (Packard, Meriden, Conn.) for 3 to 4 days. Optical densities (OD) were obtained using the Cyclone Storage Phosphor System and OptiQuant (version 3.0) acquisition and analysis software (Packard). Genes that were expressed at least 2-fold above background were included in the quantification analyses, and were normalized against the glyceraldehyde-3-phosphate dehydrogenase (GAPDH) housekeeping gene, which, by comparison to genomic DNA standards on the arrays, was found not to be altered by the experimental treatments. Data are expressed as ratios of the normalized densitometric light units (DLU) of the treated (rsHLA-G1 or rsHLA-G2) to control (PBS) messages.

### EXAMPLE 10

This example demonstrates the effect of HLA-G on the immune response to grafted tissues.

Materials and Methods

Three groups of subjects undergoing a tissue graft will be tested for endogenous levels of soluble HLA-G prior to the graft. Once the graft is complete, the subjects will once again have endogenous levels of soluble HLA-G determined. Additionally, the first group subjects will be administered a first amount of rsHLA-G. The second group will receive a second amount of rsHLA-G and the third group of subjects will not receive any additional rsHLA-G. At predetermined times thereafter, each group which received the rsHLA-G will receive additional amounts coinciding with the first amount administered to each group. The subj ects will be tested periodically to determine the levels of rsHLA-G circulating in their bloodstream and subjects which reject the grafts will be recorded together with a current rsHLA-G measurement.

### EXAMPLE 11

This example measures IL-10 production in decidual macrophages after treatment with rsHLA-G.

Materials and Methods

Decidual macrophages were isolated from extraplacental membranes by enzyme digestion and gradient centrifugation. Fresh placenta was obtained and decidual tissue was scraped away from the chorionic membrane, minced, rinsed of blood, and incubated in digestion solution (100×Penicillin/Streptomycin, 2 mM HEPES, 30 mM Sodium Bicarbonate, 10 mg/ml BSA, 200U/ml collagenase, lmg/ml hyaluronidase, 150 μg/ml DNAse in HBSS) at 37° C. for 1 hour. Cell digest was passed through cotton gauze and a 70 μm nylon mesh. Cells were collected by centrifugation (1200 rpm for 10 min.), washed with fresh medium (RPMI 1640, 10% FCS, 2 mM L-Gln, 1×Penn/Strep), and resuspended. Cells were layered over an equivalent volume of Histopaque 1077 and centrifuged at 400×g for 40 min. at room temperature. Interface cells were collected, washed three times in culture medium, and counted by Trypan blue dye exclusion.

For the cell culture and treatments, decidual cells were incubated overnight at 37° C./5% CO₂ for 18 hours in cell culture medium (RPMI 1640 containing 10% FCS, Penn/Strep, L-Gln, HEPES) to allow adherence of macrophages. Non-adherent cells and supernatant was removed, adherent cells were washed once with HBSS, and fresh culture medium containing treatments was added (IFNγ from Genzyme at 100U/ml final well concentration, rsHLA-G1 and rsHLA-G2 at 1 μg/ml final well concentrations). Cells were incubated for 24 hours at 37° C./5% CO₂, and supernatants were collected for measurement of secreted IL-10.

To measure IL-10 secretion, IL-10 production was measured by enzyme-linked immunosorbant assay (ELISA) using a pre-coated anti-human IL-10 capture ELISA kit from Pierce-Endogen. All kit procedures were followed, including the construction of a standard curve and duplicate sample measurements.

Next, Immunocytochemical staining was performed. Cytospins of freshly isolated cells were made by spinning 50,000 cells at 500 rpm for 3 min. in medium containing 50% FCS. Samples were fixed in acetone, rinsed, and blocked using 10% normal horse serum in a humidified container at room temperature for 1 hour. Primary antibody (mouse anti-human CD-14 (Zymed) at 5 μg/ml or mouse anti-human cytokeratin (Dako) at 8 μg/ml) was added and slides incubated as above. Slides were rinsed and secondary antibody (horse anti-mouse IgG (Vector) at 10 μg/ml) was added. Slides incubated in humidified container at room temperature for 25 minutes. Samples were blocked with 0.5% hydrogen peroxide in methanol, rinsed, and enzymatic activity was measured using streptavidin-peroxidase and AEC substrate kit from Zymed. Slides were rinsed, fixed, mounted and photographed.

Results and Discussion:

Isolation and Purification of the cDNA for Soluble Isoforms of HLA-G

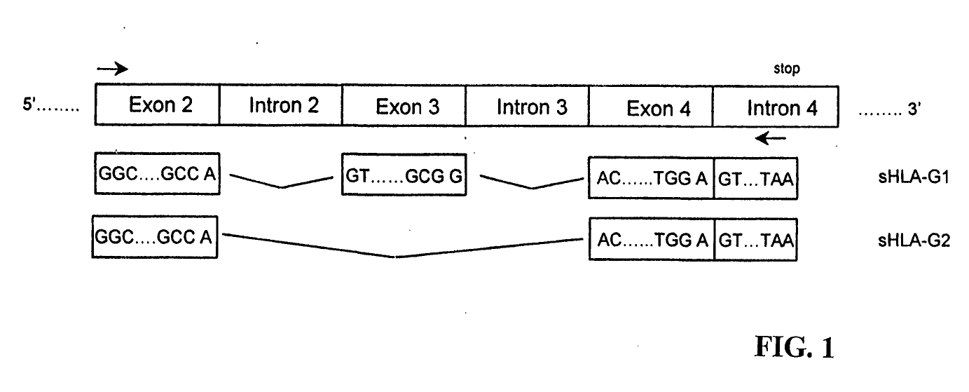

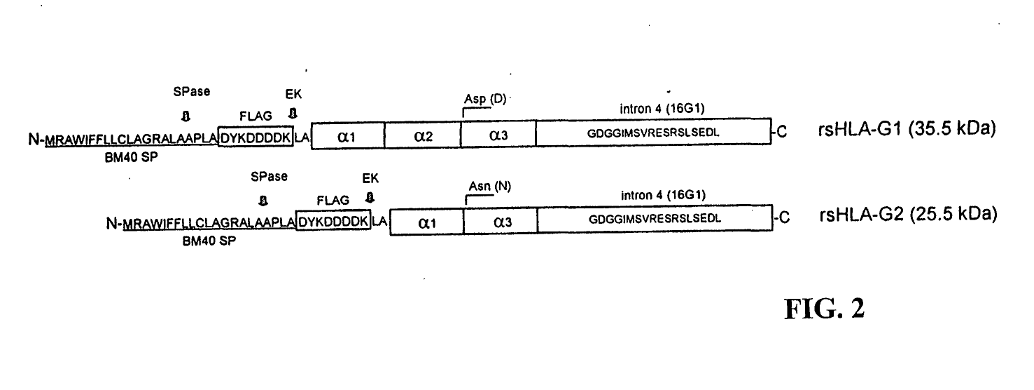

As shown by FIG. 1, the sequences for both messages derived from the S14/8.3 cells encoding sHLA-G1 and sHLA-G2 aligned exactly with the expected sequences for the coding regions for the α1, α2, α3 domains and intron 4 retaining transcripts when compared with HLA 6.0. The schematic representation of the HLA-G gene shown in FIG. 1 depicts the two alternatively spliced transcripts which retain intron 4, sHLA-G1 and sHLA-G2. The exon 4 coding region reads into intron 4 a total of 21 amino acids, then the stop codon prevents further reading of the transmembrane domain, thereby rendering these proteins soluble. The alternative splicing implies another difference between sHLA-G1 and sHLA-G2. This difference is depicted by the change in the first amino acid for the α3 domain in sHLA-G2, GAC→AAC (resulting in a change from D to N). The arrows in FIG. 1 indicate the position for the primers used in the second PCR to obtain the blunt ended cDNA fragment for cloning, as described above. The overall sequences were exactly as expected from the splicing of the messages, with sHLA-G1 (SEQ ID NO. 7) containing α1 (SEQ ID NO. 3), α2 (SEQ ID NO. 4), α3 (SEQ ID NO. 5) and the 21 amino acids encoded by intron 4 (SEQ ID NO. 6), and sHLA-G2 (SEQ ID NO. 8) being the same except for the lack of the α2 domain. It was noted that the first amino acid of the α3 domain in sHLA-G2 was asparagine instead of aspartic acid (see FIG. 2). This finding was consistent in several clones and confirms earlier theories. FIG. 2 which is a schematic representation of the recombinant products expected illustrates that both proteins (rsHLA-G1 and rsHLA-G2) both contain the same N-terminus sequence, the BM40 signal peptide for secretion of the protein into the culture media (which is released by the signal peptidase, SPase), the Flag peptide for identification and immuno-affinity purification and the enterokinase to release the Flag peptide. The C-terminus is common for both proteins containing the 21 amino acids encoded by the intron 4, which is recognized by the mouse monoclonal antibody 16G1. The expected molecular weight for each secreted protein is also provided in this figure.

Recombinant Soluble HLA-G Production

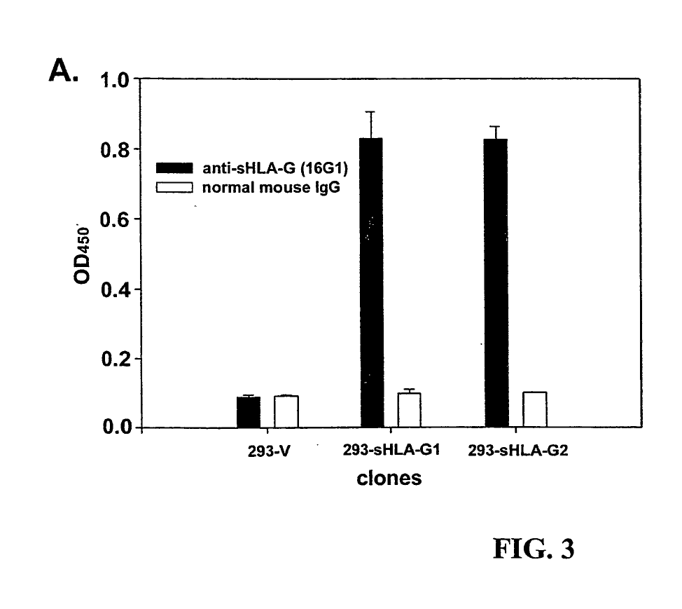

After transfection of HEK293 cells with sHLA-G constructs derived from S14/8.3 cells stably transfected clones were selected by resistance to G418. High-producing colonies were selected by limiting dilution analysis and evaluation of their production of rsHLA-G into the culture media was done using an ELISA as described in Example 2. FIG. 3 shows the results of an ELISA for rsHLA-G using the anti-sHLA-G antibody 16G1. In this figure, 293-V gives the results for the culture media supernatants from HEK293 cells stably transfected with the empty pRC/CMVBM40 vector, while 293-sHLA-G1 and 293-sHLA-G2 give the results for the soluble constructs. Each of these (293-V,293-sHLA-G1, and 293-sHLA-G2) wereusedto coat a 96 well plate and an ELISA was performed as described above. The negative control, HEK293 cells stably transfected with the empty vector, did not produce detectable reactivity for sHLA-G whereas clones carrying the construct for sHLA-G1 and -G2 secreted high levels of soluble proteins into the culture media.

Purification, Detection by Immunoblotting, and Evidence for Glycosylation of the Recombinant Soluble HLA-G1 and HLA-G2

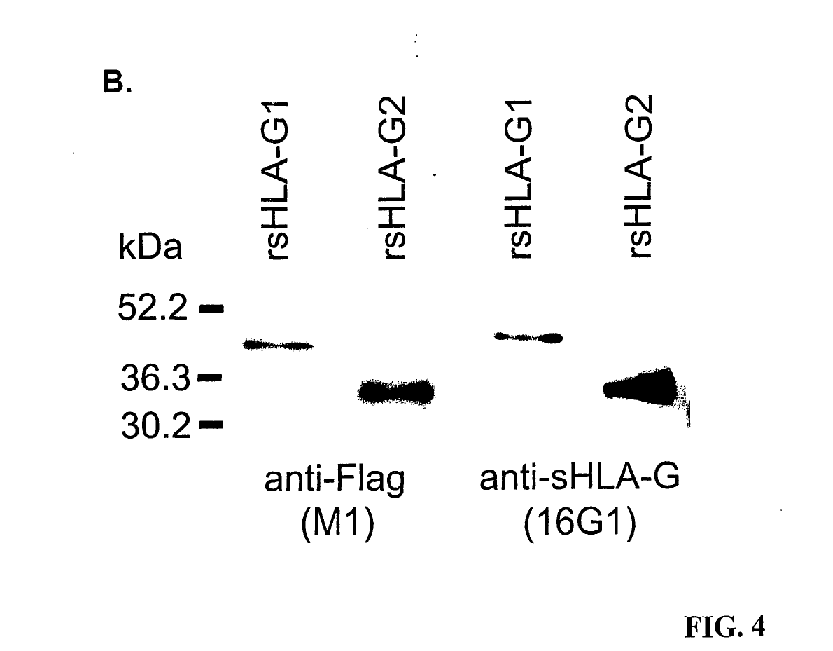

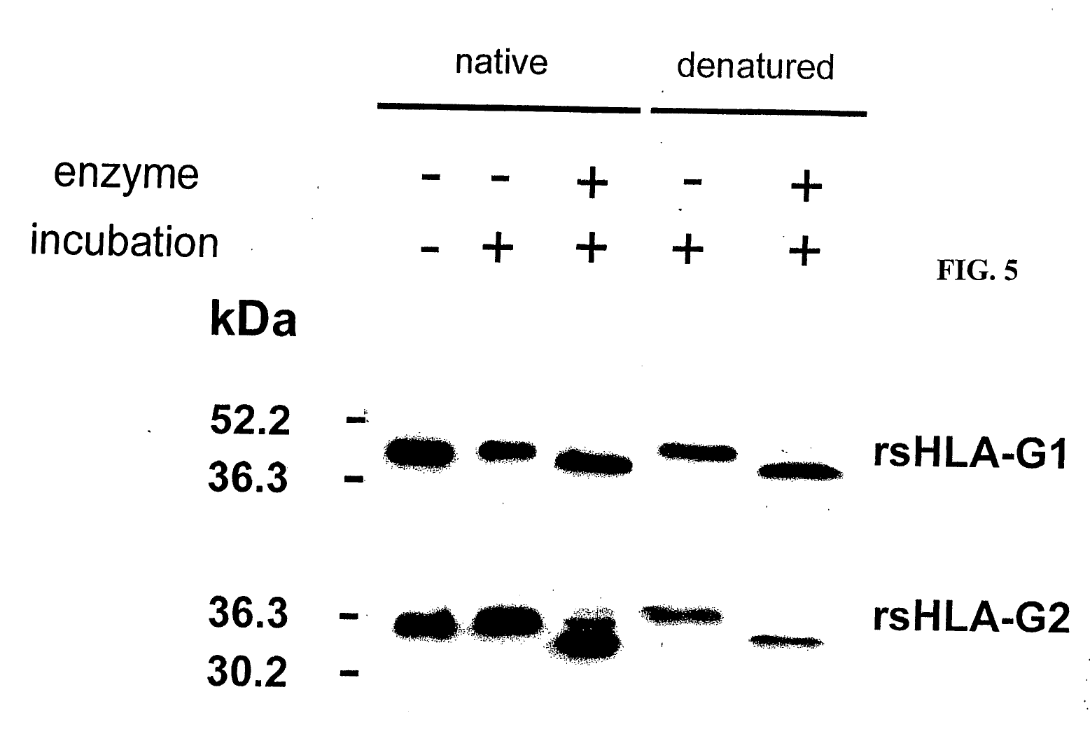

Culture supernatants from the transfected cells were purified using affinity gel chromatography. A typical yield was about 60 μg of protein per 500 ml of culture medium with purity greater than 90% as determined by Coomassie blue gel staining. Antibody 16G1 as well as the anti-Flag M1 antibody recognized the recombinant proteins by immunoblot (FIG. 4). To produce FIG. 4, immunoaffinity purified recombinant soluble HLA-G (100 ng) was detected by immunoblotting using the monoclonal anti-sHLA-G antibody, 16G1, and the Flag-M1 antibody. The apparent molecular weights of bothproteins observed by SDS-PAGE were higher (41 kDa for rsHLA-G1 and 34 kDa for rsHLA-G2) than the calculated molecular weights, which are 35.5 kDa for sHLA-G1 and 25.5 kDa for sHLA-G2 considering cleavage of the signal peptide. To investigate whether the migration patterns of the recombinant proteins were due to glycosylation, enzymatic de-glycosylation of the purified recombinant proteins under native and denaturing conditions was performed. After enzymatic digestion of the asparagine-linked (N-linked) and serine or threonine-linked (O-linked) oligosaccharides, shifts to faster migration were seen for both proteins, (FIG. 5) thus demonstrating that both rsHLA-G1 and rsHLA-G2 are glycosylated. No apparent differences for rsHLA-G1 were seen in the digestion whether the protein was under native or denaturing conditions before the treatment. For rsHLA-G2 denaturing condition was optimal for complete digestion of the oligosaccharides (FIG. 5).

Recombinant sHLA-G1 but not sHLA-G2 Binds β2m

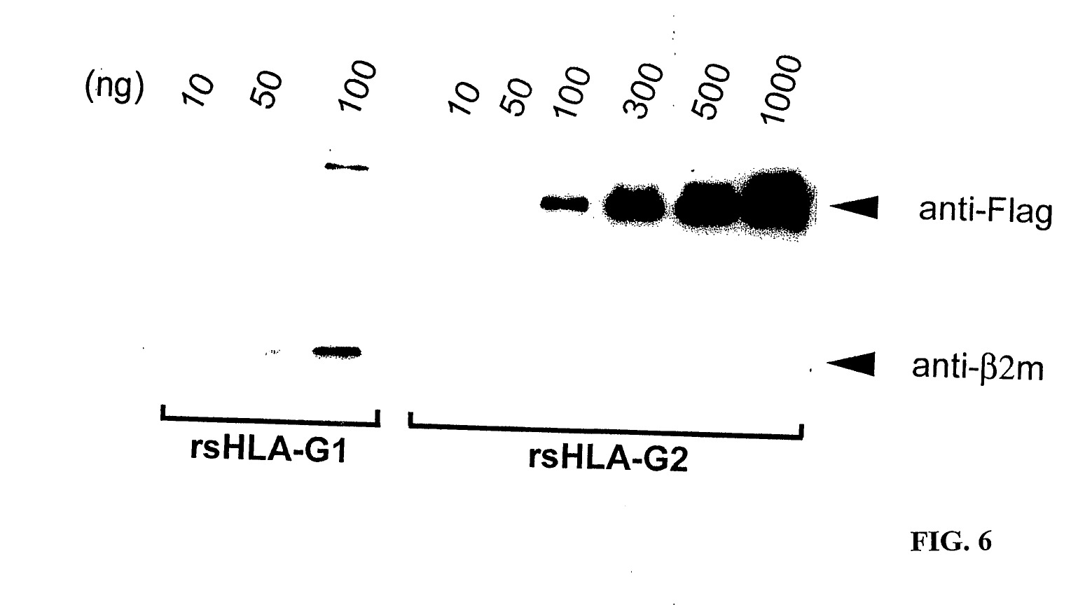

Human leukocyte antigens are heterodimeric molecules comprised of glycosylated heavy chains non-covalently associated with a 12 kDa non-polymorphic light chain called β2m. PurifiedrsHLA-G1 (10 to 100 ng) and rsHLA-G2 (10 to 1000 ng) were separated on 15% SDS-PAGE, transferred to nitrocellulose membranes and immuno-detected with anti-Flag M1 antibody or anti-β2m, as shown in FIG. 6. Only rsHLA-G1 was positive for β2m. Recombinant sHLA-G2 failed to yield a positive signal for β2m and was negative even when high amounts of the protein were used (up to 1 μg) (FIG. 6). To produce the immunoblot of FIG. 6, purified recombinant sHLA-G1 (10-100 ng) and purified recombinant sHLA-G2 (10-1000 ng) were separated using 15% PAGE-SDS and electrotransferred to nitrocellulose membranes. One blot was detected with anti-Flag-M1 antibody and the other with a mouse anti-β2m. This experiment provides evidence that sBLA-G2 is produced as free alpha chains, while sHLA-G1 is bound to β2m.

Recombinant Soluble HLA-G1 and -G2 Differentially Regulate Gene Expression in Resting and IFN-γ-Stimulated Human Blood Mononuclear Cells.

To assess whether the recombinant sHLA-G1 and sHLA-G2 were biologically functional, an array of 375 genes was screened for expression by untreated or rsHLA-G treated cells. Table 1 summarizes the effects on the pattern of gene expression from a representative experiment in which resting or IFN-γ-stimulated PBMC from a single donor were treated with either PBS, rsHLA-G1 or rsHLA-G2 under identical conditions.

TABLE 1

| | No IFN | | IFN | |
|---|---|---|---|---|
| | rsHLA-G1 | rsHLA-G2 | rsHLA-G1 | rsHLA-G2 |
| Total | 21 | 83 | 65 | 83 |
| Increased | 3 (14%) | 8 (10%) | 21 (32%) | 40 (48%) |
| Unchanged | 18 (86%) | 72 (87%) | 42 (65%) | 38 (46%) |
| Decreased | 0 | 3 (4%) | 2 (3%) | 5 (6%) |

To obtain the data contained in Table 1, peripheral blood mononuclear cells were cultured for 6 hours in the presence or absence of IFN-γ (100 U/ml) and rsHLA-G1 (1 μg/ml), rsHLA-G2 (1 μg/ml), or PBS (control). Relative mRNA expression of 375 genes were analyzed by cytokine array, quantified by phosphorimage analysis, and normalized against a housekeeping gene, glyceraldehyde-3-phosphate dehydrogenase. Results in the table are for the total number of messages detected (Total), and number (percent) of those messages with treatment:control ratios greater than 1.5 (Increased), 1.4 to 0.6 (Unchanged), or below 0.5 (Decreased).

Both rsHLA-G1 and rsHLA-G2 altered gene expression in resting as well as IFN-γ-stimulated cells. The effect of the recombinant proteins was much more profound on stimulated cells, as the percentages of genes altered by rsHLA-G1 and -G2 is higher in IFN-γ-treated cells than in resting cells. The genes that were altered by rsHLA-G1 and -G2 are listed in Table 2.

TABLE 2

| No IFN | | | | +IFN | | | |
|---|---|---|---|---|---|---|---|
| +rsHLA-G1 | | +rsHLA-G2 | | +rsHLA-G1 | | +rsHLA-G2 | |
| Enhanced | Decreased | Enhanced | Decreased | Enhanced | Decreased | Enhanced | Decreased |
| IL-1α(1.8) | | IL-1 R1 (r.9) | Caspase-1 (0.5) | IL-3 Rα (26.3) | CD6 (0.5) | IL-8 (8.7) | CD8α (0.5) |
| Endothelin-2 (1.6) | | FGF-11 (2.1) | CAD-12 (0.4) | Bob (19.2) | CD8α (0.3) | MGP-2 (5.0) | NGF R (0.5) |
| IL-1 β(1.5) | | Flt-3/Flk-2 R (2.0 | Integrin-(β1 (0.3) | EBAF (12.3) | | IL-4 Ra (4.7) | TRAIL (0.3) |
| | | NGF R (1.9) | | Flt-3/Flk-2 R (8.7) | | April (4.4) | IL-1 R1 (0.2) |
| | | Endothelin-2 (1.7) | | Angiopoetin-1 (8.3) | | MCP-1 (3.7) | FGF-11 (0.1) |
| | | EMMPRIN (1.6) | | Pleiotrophin 95.7) | | EphA-4 (3.6) | |
| | | MIC-1 (1.5) | | CAD-11 (3.60 | | TRAIL R1 (3.4) | |
| | | GFRα3 (1.5) | | IGF-11 (3.6) | | 4-IBBL (3.4) | |
| | | | | Integrin-β1 (3.0) | | MIG (3.2) | |
| | | | | IFN-1α/β Ra (2.7) | | Lymphotoxin β (3.1) | |
| | | | | Integrin-β4 (2.6) | | GRO-γ (2.9) | |
| | | | | IL-1α (2.4) | | GFG basic (2.8) | |
| | | | | Integrin-αE (2.1) | | PBEF (2.8) | |
| | | | | Integrin-β7 (1.8) | | IL-1β (2.7) | |
| | | | | TRAIL (1.8) | | CAD-12 (2.6) | |
| | | | | TIMP-1 (1.7) | | cysteine-rich GFG R | |
| | | | | | | (2.4) | |
| | | | | Caspase-1 (1.6) | | Caspase-1 (2.3) | |
| | | | | NGF R (1.5) | | PD-ECGF (2.2) | |
| | | | | Endothelin-2 (1.5) | | ChemR23 (2.2) | |
| | | | | IL-4 Rα(1.5) | | Integrin-α1 (2.1) | |
| | | | | IL-7 Rα | | IP-10 (2.1) | |
| | | | | (1.5) | | | |
| | | | | | | TGF-β R11 (2.1) | |
| | | | | | | TNF-α (1.9) | |
| | | | | | | IFN-γ R1 (1.8) | |
| | | | | | | IL-7 Rα (1.8) | |
| | | | | | | EphA7 (1.8) | |
| | | | | | | Integrin-β7 (1.7) | |
| | | | | | | CD30L (1.7) | |
| | | | | | | Flt-3/Flk-2 R (1.6) | |
| | | | | | | ICAM-1 (1.6) | |
| | | | | | | IL-10 (1.6) | |
| | | | | | | CD64 (1.6) | |
| | | | | | | Integrin-β2 (1.6) | |
| | | | | | | CD27 (1.6) | |
| | | | | | | MIP-1β (1.5) | |
| | | | | | | TACE (1.5) | |

From these results it can be seen that rsHLA-G1 and rsHLA-G2 had both overlapping and divergent effects on the cells. IL-1α and Flt-3/Flt-2R were enhanced by both rsHLA-G1 and rsHLA-G2 in resting and stimulated cells, respectively, whereas the recombinant proteins differentially affected NGF Rand TRAIL message in IFN-γ-stimulated cells. In other cases, the effect on gene expression was dependent on the activation state of the cells. For example, expression of IL-1RI, FGF 11, and NGF R mRNAs were enhanced by rsHLA-G2 in resting cells, but decreased in stimulated cells; for caspase-1 and CAD12, the opposite was true.

In the cases of HLA-G affecting tissue grafts, the presence of endogenous circulating HLA-G had a direct effect on the tolerance exhibited by the immune system of each individual. When supplemented with rsHLA-G, tolerance of the graft will improve and therefore the percentage of successful grafts will rise.

This comprises the first report of cloning and expression of the two soluble isoforms of HLA-G. Conserved transcript sequences and deduced sequences of the recombinant proteins were essentially identical to those reported or predicted for HLA class I antigens. For example, both of the cysteine pairs found in the α2 and α3 domains of other class I antigens were present in identical positions in the deduced amino acid sequence for rsHLA-G1. Further, a single N-linked glycosylation site located in the α1 region (asparagine 86) and the CD8 association site(alanine 245) as well as the nine residues involved in the peptide recognition sites were present as expected in rsHLA-G. RNA splicing seemed to be identical between the sequences encoding membrane-bound isoforms and the rsHLA-G1 and rsHLA-G2 produced using the methods of this invention. It has been reported that the first amino acid of the α3 domain in membrane-bound HLA-G2 is asparagine. This is in contrast to membrane-bound HLA-G1 in which aspartic acid is encoded. It was determined that the rsHLA-G1 cDNA sequence encodes aspartic acid and that the rsHLA-G2 sequence encodes asparagine. Moreover, the rsHLA-G proteins gave every evidence of correct glycosylation. Interestingly, rsHLA-G2 was more efficiently deglycosylated under denaturing conditions than under native conditions, suggesting that rsHLA-G2 may be folded in a manner that restricts access of the glycolytic enzymes.

Most HLA class I molecules are heterodimers composed of an alpha chain with three extracellular domains non-covalently associated with the invariant 12 kDa β2m light chain. Ithas been suggested that four regions of the α chain, one in each α1 and α2 and two in α3 were contact points between the α chain and β2m. If one of these regions is not present one might predict that α2m would not bind to the a chain. Consistent with this prediction, the present invention determined that purified rsHLA-G1 bound β2m, but, lacking the α2 domain, rsHLA-G2 did not. These findings differ from earlier reports that membrane-bound HLA-G2 binds β2m. One potential explanation for this discrepancy would be that β2m became dissociated from the recombinant sHLA-G2 during the purification procedure. However, this is unlikely because all purification steps were performed with proteins in the native state.

How the absence of the light chain might influence the structure and/or function of sHLA-G2 remains to be determined. However, associations between light chains and heavy chains are believed to be required for efficient surface expression of the α chain, and β2m binding may also affect the conformation of the α chain. Whether peptide binding would be influenced is uncertain; peptide binding to the α chain appears to precede association with β2m. Certainly, thymic development would be affected; β2m, previously known asthymotaxin, is chemotactic and is known to influence precursor T cell colonization of the thymus. β2m has also been identified as an apoptosis-inducing factor in lymphoid cells. It is therefore of note that partially purified sHLA-G1 has been associated with killing activated T-cells via the Fas/Fas ligand pathway, and with other immunomodulatory functions.

No studies using soluble HLA-G2 have been reported even though it is abundantly clear that HLA-G2 and perhaps also others of the smaller isoforms have important biological functions. Women with a homozygous deletion which precludes production of HLA-G1 isoforms carry pregnancies to term, suggesting that HLA-G2 or other isoforms may substitute for HLA-G1. Further studies suggest that maternal sera contains soluble HLA-G, and the present invention suggested that the most prominent isoform is likely to be HLA-G2. This was not definitive because antibodies distinguishing HLA-G1 and HLA-G2 heavy chains are not available at present. An alternative explanation for this finding was that only free heavy chains (both sHLA-G1 and sHLA-G2) circulate in mothers. Soluble HLA-G has also been reported in organ transplantation subjects. In a limited number of subjects, high levels of sHLA-G appear to be associated with a low incidence of acute and chronic graft rejection. To initiate elucidation of the mechanisms underlying a role for sHLA-G in feto-maternal tolerance and graft acceptance, cytokine gene array technology using human blood mononuclear cells was performed. The results demonstrate conclusively that the rsHLA-G proteins have biological activity and suggest that the two isoforms may have both redundant and non-redundant functions. Furthermore, the experiments indicate that the recombinant proteins exert differential biological activity depending on the activation state of the target cells; IFN-γ-stimulated cells were much more susceptible to gene regulation byrsHLA-G1 and -G2 than resting cells. The particular genes that were affected by the soluble proteins suggest that they function in regulation of chemokine and chemokine receptor expression, as well as expression of cell adhesion molecules. Additional studies to determine which types of leukocytes respond to the rsHLA-G molecules and to define receptors for rsHLA-G1 and -G2 are in progress. Regarding the potential for signaling, receptors for HLA-G1 have been identified on monocytes, lymphocytes, and natural killer cells but receptors responsible for the actions of sHLA-G2 have not been identified.

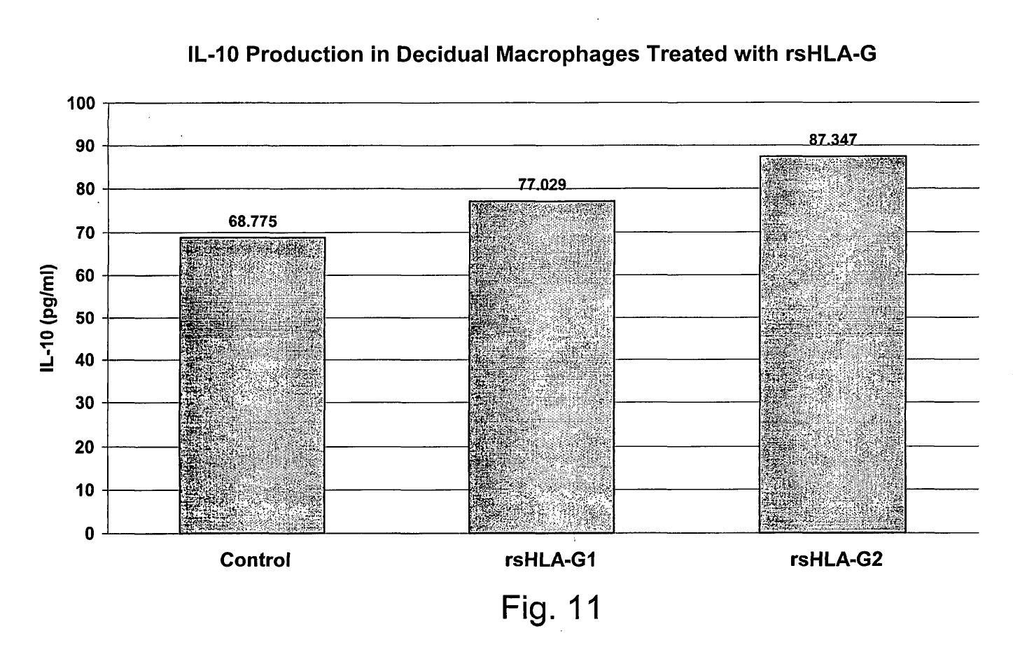

FIG. 11 illustrates IL-10 production in decidual macrophages treated with rsHLA-G. Decidual cells were isolated from extraplacental membranes and allowed to adhere on a plastic well-bottomed for 18 hours. Non-adherent cells and supernatant were removed and treatments were added as follows: control(PBS), rsHLA-G1 (1 μg/ml), rsHLA-G2 (1 μg/ml). Cells incubated for 24 hours and supernatants were collected for capture ELISA. The values shown are averages of duplicate wells.

For the immunocytochemical staining of cytospins, the results from freshly isolated decidual cells showed a majority of monocyte/macrophages with some cytotophoblasts.

In summary, the present invention reports the production of biologically active recombinant proteins derived from sequences encoding two soluble isoforms of HLA-G, i.e., rsHLA-G1 and rsHLA-G2. These proteins should be highly useful in defining the targets of the isoforms and determining their biological functions, which may include immunomodulation during pregnancy and other instances of tissue grafting and organ transplantation.

I claim:

1. A recombinant protein, said protein comprising:
a first amino acid sequence having at least 70% sequence homology with SEQ ID NO. 3;
a second amino acid sequence linked with said first amino acid sequence, said second amino acid sequence having at least 70% sequence homology with SEQ ID NO. 5; and
a third amino acid sequence linked with said second amino acid sequence, said third amino acid sequence having at least 70% sequence homology with SEQ ID NO. 6.

2. The recombinant protein of claim 1, further comprising a signal peptide.

3. The recombinant protein of claim 2, said signal peptide having at least 70% sequence homology with SEQ ID NO. 1.

4. The recombinant protein of claim 2, said signal peptide being BM40.

5. The recombinant protein of claim 2, said signal peptide being linked with said first amino acid sequence.

6. The recombinant protein of claim 1, further comprising a fourth amino acid sequence linked with said first amino acid sequence, said fourth amino acid sequence having at least 70% sequence homology with SEQ ID NO. 1.

7. The recombinant protein of claim 1, further comprising a fifth amino acid sequence linked with said first amino acid sequence, said fifth amino acid sequence having at least 70% sequence homology with SEQ ID NO. 2.

8. The recombinant protein of claim 1, further comprising a sixth amino acid sequence linked with said first amino acid sequence, said sixth amino acid sequence having at least 70% sequence homology with SEQ ID NO. 4.

9. The recombinant protein of claim 1, said first amino acid sequence having at least 79% sequence homology with SEQ ID NO. 3.

10. The recombinant protein of claim 1, said second amino acid sequence having at least 79% sequence homology with SEQ ID NO. 5.

11. The recombinant protein of claim 1, said third amino acid sequence having at least 79% sequence homology with SEQ ID NO. 6.

12. The recombinant protein of claim 6, said fourth amino acid sequence having at least 79% sequence homology with SEQ ID NO. 1.

13. The recombinant protein of claim 7, said fifth amino acid sequence having at least 79% sequence homology with SEQ ID NO. 2.

15. The recombinant protein of claim 8, said sixth amino acid sequence having at least 79% sequence homology with SEQ ID NO. 4.

16. The recombinant protein of claim 1, said recombinant protein being soluble in aqueous solutions.

17. The recombinant protein of claim 16, said aqueous solutions comprising at least 50% water.

18. The recombinant protein of claim 16, said aqueous solutions comprising water and at least one member selected from the group consisting of salts, detergents, and buffers.

19. The recombinant protein of claim 1, said recombinant protein altering activity in a cell, said cell selected from the group consisting of peripheral blood mononuclear cells and decidual cells.

20. A recombinant protein encoded by a gene transcript of a cell, said transcript comprising in succession from the 5′ end to the 3′ end:
a first sequence encoding the α1 domain of said protein;
a second sequence encoding the α3 domain of said protein; and
a third sequence encoding intron 4.

21. The recombinant protein of claim 20, said transcript further comprising a fourth sequence encoding the α2 domain of said protein.

22. The recombinant protein of claim 20, said transcript further comprising a fifth sequence encoding a signal peptide.

22. The recombinant protein of claim 20, said signal peptide being cleaved from said recombinant protein in said cell.

24. The recombinant protein of claim 20, said transcript further comprising a sixth sequence encoding a purification-assisting peptide.

25. The recombinant protein of claim 24, said purification-assisting peptide being cleaved from said recombinant protein in said cell.

26. The recombinant protein of claim 24, said purification-assisting peptide having at least 70% sequence homology with SEQ ID NO. 2.

27. The recombinant protein of claim 20, said cell being a eukaryotic cell.

28. The recombinant protein of claim 20, said cell being a transfected mammalian cell.

29. The recombinant protein of claim 28, said transfected mammalian cell comprising mammalian cells transfected with an expression vector for sHLA-G1 and sHLA-G2.

30. The recombinant protein of claim 29, said expression vector comprising a sequence encoding a protein having at least 70% sequence homology with a sequence selected from the group consisting of SEQ ID NO. 7 and SEQ ID NO. 8.

31. The recombinant protein of claim 20, said cell being selected from the group consisting of HEK293 cells, human trophoblast tumor cells, and Chinese hamster ovary cells.

32. The recombinant protein of claim 20, said cell being and HEK293 cell.

33. The recombinant protein of claim 20, said recombinant protein being secreted from said cell.

34. The recombinant protein of claim 20, said recombinant protein being soluble in aqueous solutions.

35. The recombinant protein of claim 34, said aqueous solutions comprising at least 50% water.

36. The recombinant protein of claim 34, said aqueous solutions comprising water and at least one member selected from the group consisting of salts, detergents, and buffers.

37. A recombinant protein having at least 70% sequence homology with an amino acid sequence, said amino acid sequence comprising SEQ ID NO. 6 and at least one sequence selected from the group consisting of SEQ ID NOS. 3-5.

38. The recombinant protein of claim 37, said amino acid sequence further comprising a sequence selected from the group consisting of SEQ ID NO. 1, SEQ ID NO. 2 and combinations thereof.

39. The recombinant protein of claim 37, said recombinant protein having at least 70% sequence homology with a first amino acid sequence comprising, in succession SEQ ID NO. 3, SEQ ID NO. 5, and SEQ ID NO. 6.

40. The recombinant protein of claim 39, further comprising a second amino acid sequence having at least 70% sequence homology with SEQ ID NO. 1, said second amino acid sequence being linked to said first amino acid sequence.

41. The recombinant protein of claim 40, further comprising a third amino acid sequence having at least 70% sequence homology with SEQ ID NO. 2, said third amino acid sequence being linked to said first amino acid sequence.

42. The recombinant protein of claim 37, said protein being expressed by eukaryotic cells.

43. The recombinant protein of claim 42, said eukaryotic cells comprising mammalian cells.

44. The recombinant protein of claim 43, said eukaryotic cells being selected from the group consisting of HEK293 cells, human trophoblast tumor cells, and Chinese hamster ovary cells.

45. The recombinant protein of claim 43, said eukaryotic cells comprising HEK293 cells.

46. The recombinant protein of claim 37, said amino acid sequence being SEQ ID NO. 7.

47. The recombinant protein of claim 37, said amino acid sequence being SEQ ID NO. 8.

48. The recombinant protein of claim 37, said recombinant protein being soluble in aqueous solutions.

49. The recombinant protein of claim 48, said aqueous solutions comprising at least 50% water.

50. The recombinant protein of claim 48, said aqueous solutions comprising water and at least one member selected from the group consisting of salts, detergents, and buffers.

51. A recombinant expression vector, said vector expressing a recombinant protein, said recombinant protein having at least 70% sequence homology with an amino acid sequence, said amino acid sequence comprising SEQ ID NO. 6 and at least one sequence selected from the group consisting of SEQ ID NOS. 3-5.

52. The recombinant expression vector of claim 51, said amino acid sequence further comprising a sequence selected from the group consisting of SEQ ID NO. 1, SEQ ID NO. 2 and combinations thereof.

53. The recombinant expression vector of claim 51, said recombinant protein having at least 70% sequence homology with a first amino acid sequence comprising, in succession SEQ ID NO. 3, SEQ ID NO. 5, and SEQ ID NO. 6.

54. The recombinant expression vector of claim 53, said recombinant protein further comprising a second amino acid sequence having at least 70% sequence homology with SEQ ID NO. 1, said second amino acid sequence being linked to said first amino acid sequence.

55. The recombinant expression vector of claim 54, said recombinant protein further comprising a third amino acid sequence having at least 70% sequence homology with SEQ ID NO. 2, said third amino acid sequence being linked to said first amino acid sequence.

56. The recombinant expression vector of claim 51, said vector being transfected into eukaryotic cells.

57. The recombinant expression vector of claim 56, said transfected eukaryotic cells being stable.

58. The recombinant expression vector of claim 51, said vector being transfected into mammalian cells.

59. The recombinant expression vector of claim 51, said vector being transfected into a cell selected from the group consisting of HEK293 cells, human trophoblast tumor cells, and Chinese hamster ovary cells.

60. The recombinant expression vector of claim 59, said cell being an HEK293 cell.

61. The recombinant expression vector of claim 56, said amino acid sequence being expressed by said transfected cells.

62. The recombinant expression vector of claim 56, said amino acid sequence being secreted from said cell.

63. The recombinant expression vector of claim 51, said recombinant protein being soluble in aqueous solutions.

64. The recombinant expression vector of claim 63, said aqueous solutions comprising at least 50% water.

65. The recombinant expression vector of claim 63, said aqueous solutions comprising water and at least one member selected from the group consisting of salts, detergents, and buffers.

66. A method of promoting acceptance of a fetus by the immune system of an individual comprising the steps of:
administering a recombinant protein to the individual; and
altering cytokine production in the individual by contacting a target cell within the individual with said recombinant protein.

67. The method of claim 66, said recombinant protein having at least 70% sequence homology with an amino acid sequence, said amino acid sequence comprising SEQ ID NO. 6 and at least one sequence selected from the group consisting of SEQ ID NOS. 3-5.

68. The method of claim 66, said amino acid sequence further comprising a sequence selected from the group consisting of SEQ ID NO. 1, SEQ ID NO. 2 and combinations thereof.

69. The method of claim 66, said recombinant protein comprising an amino acid sequence having at least 70% sequence homology with a sequence selected from the group consisting of SEQ ID NOS. 7 and 8.

70. The method of claim 66, said recombinant protein being soluble in aqueous solutions.

71. The method of claim 70, said aqueous solutions comprising at least 50% water.

72. The method of claim 70, said aqueous solutions comprising water and at least one member selected from the group consisting of salts, detergents, and buffers.

73. The method of claim 66, said altering cytokine production including the step of altering signal transduction in said target cell.

74. The method of claim 73, said target cell binding soluble HLA-G.

75. The method of claim 73, said target cell being selected from the group consisting of leukocytes and decidual cells.

76. A method of modulating an individual's immune tolerance to foreign tissue comprising the steps of:
administering a recombinant protein to the individual, said recombinant protein binding to a target cell; and
causing said recombinant protein to modulate gene expression of said target cell.

77. The method of claim 76, said target cell binding soluble HLA-G.

78. The method of claim 76, said target cell being selected from the group consisting of leukocytes and decidual cells.

79. The method of claim 76, said recombinant protein having at least 70% sequence homology with an amino acid sequence, said amino acid sequence comprising SEQ ID NO. 6 and at least one sequence selected from the group consisting of SEQ ID NOS. 3-5.

80. The method of claim 79, said amino acid sequence further comprising a sequence selected from the group consisting of SEQ ID NO. 1, SEQ ID NO. 2 and combinations thereof.

81. The method of claim 76, said recombinant protein comprising an amino acid sequence having at least 70% sequence homology with a sequence selected from the group consisting of SEQ ID NOS. 7 and 8.

82. The method of claim 76, said recombinant protein being soluble in aqueous solutions.

83. The method of claim 82, said aqueous solutions comprising at least 50% water.

84. The method of claim 82, said aqueous solutions comprising water and at least one member selected from the group consisting of salts, detergents, and buffers.

85. The method of claim 76, said modulating target cell gene expression including the step of altering signal transduction in said target cell.

86. A method of obtaining recombinant soluble HLA-G protein comprising the steps of:
transfecting a first cell containing the gene encoding HLA-G protein;
isolating RNA from said transfected cell;
preparing cDNA from said isolated RNA;
amplifying said cDNA by performing PCR on said cDNA;
selecting for amplified cDNA containing selected portions of the cDNA encoding for HLA-G;
ligating said amplified cDNA containing said selected portions into a vector to produce a recombinant expression vector;
transfecting a second cell with said recombinant expression vector; and
expressing said selected portions using said transfected second cell.

87. The method of claim 86, said first cell comprising a mouse fibroblast cell line.

88. The method of claim 86, said first cell comprising an S14/8 mouse fibroblast cell.

89. The method of claim 86, said preparing step including the step of using reverse transcriptase.

90. The method of claim 89, said reverse transcriptase comprising Moloney Murine Leukemia Virus reverse transcriptase.

91. The method of claim 86, said PCR using a forward primer comprising a sequence having at least about 70% sequence homology with SEQ ID NO. 9.

92. The method of claim 86, said PCR using a reverse primer comprising a sequence having at least about 70% sequence homology with SEQ ID NO. 10.

93. The method of claim 86, said selected portions including sequences encoding for sequences having at least 70% sequence homology with SEQ ID NO. 6 and at least one sequence having at least 70% sequence homology with a sequence selected from the group consisting of SEQ ID NOS. 3-5.

94. The method of claim 86, said ligating step utilizing T4 DNA ligase.

95. The method of claim 86, said vector being linearized.

96. The method of claim 86, said vector being the expression vector pcDNA3.1HisC.

97. The method of claim 86, said second cell being a eukaryotic cell.

98. The method of claim 86, said second cell being a mammalian cell.

99. The method of claim 98, said mammalian cell being selected from the group consisting of HEK293 cells, human trophoblast tumor cells, and Chinese hamster ovary cells.

100. The method of claim 86, said expressed selected portions comprising recombinant soluble HLA-G protein.

101. The method of claim 78, said recombinant soluble HLA-G protein being selected from the group consisting of HLA-G1, HLA-G2, and combinations thereof.

102. The method of claim 86, said recombinant soluble HLA-G protein having at least 70% sequence homology with a sequence selected from the group consisting of SEQ ID NO. 7 and SEQ ID NO. 8.

103. A method of producing recombinant soluble HLA-G comprising the steps of:
transfecting a cell with a recombinant expression vector, said vector including a sequence encoding for intron 4 of HLA-G and at least one sequence encoding for a domain selected from the group consisting of the α1 domain, the α2 domain, the α3 domain and combinations thereof; and
expressing said sequence of said recombinant expression vector in said cell.
